(19) United States
(12) Patent Application Publication
(10) Pub. No.: US 2024/0009239 A1
(43) Pub. Date: Jan. 11, 2024

(54) THERAPEUTIC TARGETING OF MESOTHELIN IN ACUTE MYELOID LEUKEMIA WITH CHIMERIC ANTIGEN RECEPTOR T CELL THERAPY

(71) Applicant: FRED HUTCHINSON CANCER CENTER, Seattle, WA (US)

(72) Inventors: Soheil Meshinchi, Seattle, WA (US); Quy Le, Seattle, WA (US); Rhonda Ries, Seattle, WA (US)

(73) Assignee: FRED HUTCHINSON CANCER CENTER, Seattle, WA (US)

(21) Appl. No.: 18/251,871

(22) PCT Filed: Nov. 3, 2021
(86) PCT No.: PCT/US2021/057955

§ 371 Date: May 4, 2023

Related U.S. Application Data
(60) Provisional application No. 63/109,815, filed on Nov. 4, 2020.

(51) Int. Cl. A61K 35/17; C07K 16/28; A61P 35/02; A61K 39/00

(52) U.S. Cl. CPC A61K 35/17; C07K 16/28; A61P 35/02; A61K 39/4613; A61K 39/4611; A61K 39/4631; A61K 39/464468; A61K 2239/17; A61K 2239/21; A61K 2239/48; A61K 2239/13

(57) ABSTRACT

In various embodiments, the present disclosure provides chimeric antigen receptors (CARs) which bind to mesothelin. The mesothelin CARs comprise an extracellular region comprising a binding domain that specifically binds to at least a portion of mesothelin, a transmembrane region, and an intracellular region comprising an effector domain or a portion or variant thereof and a costimulatory domain or a portion or variant thereof. Recombinant host cells expressing the mesothelin CARs are also provided, as well as compositions and methods of treatment, prevention, and manufacture comprising the same.

### PRIORITY CLAIM

This application claims the benefit of U.S. Provisional Patent Application No. 63/109,815, filed Nov. 4, 2020, which is incorporated herein by reference in its entirety, including drawings.

### SEQUENCE LISTING

This application contains a Sequence Listing, which was submitted in ASCII format via EFS-Web, and is hereby incorporated by reference in its entirety. The ASCII copy, created on Nov. 3, 2021, is named SequenceListing.txt and is 35 KB in size.

### TECHNICAL FIELD

Provided herein are compositions and methods related to cancer treatment and prognosis, and more specifically to treatment of such conditions with chimeric antigen receptor (CAR)-modified T cells (CAR T) expressing one or more CARs which bind to mesothelin.

### BACKGROUND

Acute myeloid leukemia (AML) is one of the most highly refractory hematologic malignancies despite intensive combination chemotherapy and bone marrow stem cell transplantation. Lack of curative treatments is in large part due to poor understanding of the disease biology and paucity of therapeutic targets.

In an effort to identify actionable targets, the largest genome, epigenome, and transcriptome profiling of AML in nearly 3000 children and young adults was recently completed. This led to the identification of a library of novel AML-restricted targets (e.g., high expression in AML, minimal-to-no expression in normal hematopoiesis). One such target was MSLN, which encodes for mesothelin, a cell surface adhesion molecule. MSLN expression in normal tissues is confined to mesothelial cells lining the pleura, pericardium, and peritoneum. In AML, MSLN is highly expressed in 30%-50% of cases in pediatric (Children Oncology Group) and adult (MD Anderson) cohorts yet is absent in normal bone marrow and peripheral blood CD34+ cells.

Accordingly, there exists a need for novel therapies directed to actionable targets such as MSLN.

### SUMMARY

The present disclosure provides chimeric antigen receptors (CARs) which bind to mesothelin. The mesothelin CARs comprise an extracellular region comprising a binding domain that specifically binds to at least a portion of mesothelin, a transmembrane region, and an intracellular region comprising an effector domain or a portion or variant thereof and a costimulatory domain or a portion or variant thereof. Recombinant host cells expressing the mesothelin CARs are also provided, as well as compositions and methods of treatment, prevention, and manufacture comprising the same.

In some aspects, provided are CARs that bind to at least one epitope of mesothelin. In some embodiments, the CAR comprises a signal peptide, a binding domain specific to mesothelin, a hinge domain, a transmembrane domain, a costimulatory domain, and/or an effector domain. In some embodiments, the signal peptide comprises a GM-CSFR signal peptide. In some embodiments, the hinge domain comprises an IgG4 hinge domain. In some embodiments, the transmembrane domain comprises a CD28 transmembrane domain. In some embodiments, the costimulatory domain comprises a 4-1BB costimulatory domain. In some embodiments, the effector domain comprises a CD3ζ effector domain.

In some embodiments, the binding domain comprises an scFv. In some embodiments, the scFv comprises a light chain variable region (V_{L}) having an amino acid sequence that is at least 75%, 80%, 85%, 90%, 91%, 92%, 93%, 94%, 95%, 96%, 97%, 98%, 99%, or 100% identical to SEQ ID NO: 1. In some embodiments, the scFv comprises a heavy chain variable region (V_{H}) having an amino acid sequence that is at least 75%, 80%, 85%, 90%, 91%, 92%, 93%, 94%, 95%, 96%, 97%, 98%, 99%, or 100% identical to SEQ ID NO: 3. In some embodiments, the scFv comprises a (G₄S)₄ linker connecting the V_{L} and the V_{H}. In some embodiments, the scFv comprises an amino acid sequence that is at least 75%, 80%, 85%, 90%, 91%, 92%, 93%, 94%, 95%, 96%, 97%, 98%, 99%, or 100% identical to any one of SEQ ID NOs: 1-3.

In some embodiments, the CAR further comprises a spacer between the hinge domain and the transmembrane domain. In some embodiments, the spacer comprises an amino acid sequence that is at least 75%, 80%, 85%, 90%, 91%, 92%, 93%, 94%, 95%, 96%, 97%, 98%, 99%, or 100% identical to SEQ ID NO: 7 or SEQ ID NO: 8.

In some embodiments, the CAR further comprises a polypeptide marker. In some embodiments, the polypeptide marker comprises a truncated form of CD19 (CD19t) comprising an amino acid sequence set forth in SEQ ID NO: 12. In some embodiments, the CD19t is separated from the CAR by a T2A sequence comprising an amino acid sequence set forth in SEQ ID NO: 11.

In some embodiments, the CAR comprises an amino acid sequence that is at least 75%, 80%, 85%, 90%, 91%, 92%, 93%, 94%, 95%, 96%, 97%, 98%, 99%, or 100% identical to any one of SEQ ID NOs: 13-15.

In some aspects, provided are isolated polynucleotides encoding the CAR according to various embodiments of the technology. In some embodiments, the polynucleotide is in a vector.

In some aspects, provided are T cells, natural killer (NK) cells, or NKT cells expressing the CAR according to various embodiments of the technology or comprising the polynucleotide according to various embodiments of the technology.

In some aspects, provided are methods of treating and/or preventing a cancer associated with mesothelin expression in a subject in need thereof, comprising administering to the subject a therapeutically effective amount of the CAR, the polynucleotide, or the T cells, NK cells, or NKT cells according to various embodiments of the technology. In some embodiments, the cancer is AML.

### BRIEF DESCRIPTION OF THE DRAWINGS

FIGS. 1A-1E. Pre-clinical efficacy of MSLN CAR T cells against MSLN-positive AML cells. FIG. 1A. Cytolytic activity of CD8 T cells transduced with MSLN CAR (CAR T) or mock transduced (Mock T) following 24-hour co-culture with Nomo-1 and 10 hours with Kasumi-1 MSLN+ and Kasumi-1 parental cells. Data presented are mean leukemia specific lysis+/−SD from triplicates at indicated effector:target (E:T) ratios. FIG. 1B. Shown are the concentrations of secreted IL-2, IFN-γ, and TNF-α in the supernatant following 24 hours of T cell/AML co-culture at 1:1 E:T ratio as measured by ELISA. Data presented are mean+/−SD. Where concentrations of cytokines are too low to discern, the number indicates the average concentration. FIG. 1C. CFSE-labeled mock transduced or CAR T cells were incubated with target cells at E:T 1:1 for 4 days. Representative flow cytometric analysis of cell proliferation (CFSE dilution) of CAR and Mock T cells. FIG. 1D. The in vivo efficacy of MSLN CAR T cells was evaluated in Nomo-1 and Kasumi-1 MSLN+ AML xenografts. 10⁶ leukemia cells were injected into NSG mice. Mock transduced or MSLN CAR T cells were infused 1 week (Nomo-1) or 2 weeks (Kasumi-1 MSLN+) post leukemia injection. Tumor burden was measured by bioluminescence IVIS imaging weekly until mice developed symptoms or until an experimental endpoint of 16 weeks post leukemia injection. FIG. 1E. Expansion of CD4 and CD8 T cell subsets was detected in the peripheral blood.

FIGS. 2A-2D. Inhibiting ADAM17-mediated MSLN shedding may enhance activity of CAR T cells. FIGS. 2A-2B. Nomo-1 cells were incubated with GM6001 (50 uM) for 48 hours. Cell surface abundance and viability were measured by flow cytometry (FIG. 2A) and shed MSLN was quantified by ELISA (FIG. 2B). FIG. 2C. Cytolytic activity of MSLN CAR CD8 T cells against Nomo-1 pre-treated with GM6001 or DMSO control for 48 hours in an 8-hour assay. Data presented are mean tumor specific lysis+/−standard deviation from 3 technical replicates at indicated E:T ratios for 3 independent experiments. FIG. 2D. Secreted levels of IFN-g (left panel) and TNF-a (right panel) were measured after 8 hours of T cell/AML co-culture at E:T 10:1 by ELISA. Data presented are mean+/−standard deviation from 3 technical replicates. Statistical significance was determined by unpaired Student's t test, assuming unequal variances. p<0.05(*), p<0.005(**), p<0.0005(***).

FIGS. 3A-3C. MSLN CAR constructs and cytolytic activity of short, intermediate, and long MSLN CAR T cells. FIG. 3A. Schematic diagram of second-generation anti-MSLN CAR constructs with varying spacer lengths. FIG. 3B. Flow cytometric analysis of AML cell lines expressing exogenous (Nomo-1) and exogenous MSLN (Kasumi-1 MSLN+) and their MSLN-negative counterparts. FIG. 3C. Cytolytic activity of short, intermediate, and long CAR and mock transduced CD8 T cells against Nomo-1 and Kasumi-1 MSLN+ target cells in 8-hour assay. Data presented are mean percent specific lysis+/−standard deviation from 3 technical replicates at indicated E:T ratios.

FIG. 4. Cytolytic activity of MSLN CAR T cells against Kasumi-1 MSLN+ cells. Cytolytic activity of CD8 CAR and mock transduced T cells derived from 2 additional donors was assayed against Kasumi-1 MSLN+ and Kasumi-1 parental cells in an 8-hour assay. Data presented are mean tumor specific lysis+/−standard deviation from 3 technical replicates at indicated E:T ratios.

FIG. 5. MSLN CAR T cells undergo enhanced proliferation in the presence of MSLN-positive AML cells. Shown is the percent of CD4 and CD8 T cells that have divided during 4-day coincubation with Nomo-1, Kasumi-1 MSLN+, and Kasumi-1 parental cells.

FIGS. 6A-6B. MSLN CAR T cells demonstrated in vivo efficacy in Kasumi-1 MSLN+ AML xenografts. FIG. 6A. Diagram illustrating the generation of luciferase-expressing Kasumi-1 MSLN+ and Kasumi-1 parental AML xenografts in NSG mice and treatment with MSLN CAR T cells and mock transduced T cells. Tumor burden was measured by bioluminescence IVIS imaging weekly until mice developed symptoms or until an experimental endpoint of 16 weeks post tumor injection. FIG. 6B. Quantification of tumor burden over time in Kasumi-1 MSLN+ and parental xenografts untreated or following injection with CAR T cells. Data are plotted relative to baseline (one day before CAR T cell injection (Day −1)). Tumor burden is shown for each mouse.

### DETAILED DESCRIPTION

The present disclosure provides chimeric antigen receptors (CARs) which bind to mesothelin (MSLN), cells expressing these CARs, and methods of using these CARs and cells expressing the same.

When expressed by a cell and bound to mesothelin expressed or shed by a target cell, the mesothelin CARs provided herein induce initiation, propagation, and/or magnification of a molecular signal in the cell, such as cytotoxicity, proliferation, and/or survival. Exemplary CARs of the present disclosure comprise (a) an extracellular region comprising a binding domain (e.g., an scFv) that specifically binds to mesothelin; (b) a transmembrane region; and (c) an intracellular region comprising an effector domain or a portion or variant thereof, and a costimulatory domain or a portion or variant thereof.

The mesothelin CARs of the present disclosure are useful in cellular immunotherapies (e.g., T cell and/or natural killer (NK) cell) for treating a disease or condition associated with mesothelin expression, such as a malignancy. In some embodiments, the malignancy is AML. In some embodiments, when administered to a subject having target cells (e.g., malignant cells) that express and/or shed mesothelin, cells expressing mesothelin CARs of the present disclosure reduce and/or suppress growth, area, volume, and/or spread of the malignant cells, eliminate (e.g., kill) malignant cells, and/or increase survival of the subject to a greater degree and/or for a longer period of time than cells that do not comprise a mesothelin CAR of the present disclosure.

In some embodiments, a T cell and/or an NK cell expressing a mesothelin CAR described herein demonstrates increased and/or sustained cell signaling, such as cytokine production and/or release, phosphorylation of one or more proteins associated with a T cell response to antigen-binding, and/or activity, such as mobilization of intracellular calcium, cytotoxic activity, secretion of a cytokine, proliferation, and/or activation following stimulation. One or more of these effects occurring in response to mesothelin (e.g., shed or expressed by a cell) binding is improved relative to a T cell and/or an NK cell that does not express a mesothelin CAR of the present disclosure.

The following description of the present disclosure is merely intended to illustrate various embodiments of the present disclosure. As such, the specific modifications discussed herein are not to be construed as limitations on the scope of the present disclosure. It will be apparent to one skilled in the art that various equivalents, changes, and modifications may be made without departing from the scope of the present disclosure, and it is understood that such equivalent embodiments are to be included herein. While the present disclosure is capable of being embodied in various forms, the description below of several embodiments is made with the understanding that the present disclosure is to be considered as an exemplification of the invention and is not intended to limit the invention to the specific embodiments illustrated.

Reference throughout this specification to “one example,” “an example,” “one embodiment,” “an embodiment,” “one aspect,” or “an aspect” means that a particular feature, structure, or characteristic described in connection with the example is included in at least one example of the present disclosure. Thus, the occurrences of the phrases “in one example,” “in an example,” “one embodiment,” “an embodiment,” “one aspect,” or “an aspect” in various places throughout this specification are not necessarily all referring to the same example, embodiment, and/or aspect.

To the extent any materials incorporated herein by reference conflict with the present disclosure, the present disclosure controls.

Headings are provided for convenience only and are not to be construed to limit the invention in any manner. Embodiments illustrated under any heading may be combined with embodiments illustrated under any other heading.

### Definitions

Unless otherwise specified, each of the following terms has the meaning set forth in this section.

The indefinite articles “a” and “an” denote at least one of the associated nouns and are used interchangeably with the terms “at least one” and “one or more.” For example, the phrase “a module” means at least one module, or one or more modules.

The conjunctions “or” and “and/or” are used interchangeably.

The term “including” is used interchangeably with the term “including, but not limited to.”

The term “about,” as used herein when referring to a measurable value such as an amount or concentration and the like, is meant to encompass variations of 20%, 10%, 5%, 1%, 0.5%, or even 0.1% of the specified amount.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein is to be understood to include any integer within the recited range, unless otherwise indicated.

A “subject” means a human, mouse, or non-human primate. A human subject can be any age (e.g., an infant, child, young adult, or adult), and may suffer from a disease, such as a cancer. In some embodiments, a subject is suffering from a relevant disease, disorder, or condition. In some embodiments, a subject is susceptible to a disease, disorder, or condition. In some embodiments, a subject displays one or more symptoms or characteristics of a disease, disorder, or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

The phrases “subject” and “patient” are used interchangeably herein.

The terms “treat,” “treating,” and “treatment” as used herein with regard to cancer refer to alleviating the cancer partially or entirely, inhibiting cancer cell growth, reducing the number of cancer cells, preventing the cancer, decreasing the likelihood of occurrence or recurrence of the cancer, slowing the progression or development of the cancer, or eliminating, reducing, or slowing the development of one or more symptoms associated with the cancer. For example, “treating” may refer to preventing or slowing the existing tumor from growing larger, preventing or slowing the formation or metastasis of cancer, and/or slowing the development of certain symptoms of the cancer. In some embodiments, the term “treat,” “treating,” or “treatment” means that the subject has a reduced number or size of tumors compared to a subject who does not receive such treatment. In some embodiments, the term “treat,” “treating,” or “treatment” means that one or more symptoms of the cancer are alleviated in a subject receiving the pharmaceutical compositions as disclosed and described herein compared to a subject who does not receive such treatment.

The terms “prevent,” “preventing,” and “prevention” as used herein mean the prevention of a disease (e.g., cancer) in a subject (e.g., in a human), including (a) avoiding or precluding the disease; (b) affecting the predisposition toward the disease; and (c) preventing or delaying the onset of and/or reduction in frequency and/or severity of at least one symptom of the disease.

As used herein, “hyperproliferative disorder” and “proliferative disorder” refer to excessive growth or proliferation as compared to a normal or undiseased cell. Exemplary hyperproliferative disorders and proliferative disorders include tumors, cancers, neoplastic tissue, carcinoma, sarcoma, malignant cells, premalignant cells.

Furthermore, “cancer” may refer to any accelerated proliferation of cells, including solid tumors, ascites tumors, blood or lymph or other malignancies; connective tissue malignancies; metastatic disease; minimal residual disease following transplantation of organs or stem cells; multi-drug resistant cancers, primary or secondary malignancies, angiogenesis related to malignancy, or other forms of cancer. In some embodiments, the cancer is AML.

The terms “peptide,” “polypeptide,” and “protein” are used interchangeably to refer to a polymer of amino acid residues, and are not limited to a minimum length, though a number of amino acid residues may be specified. Polypeptides may include amino acid residues including natural and/or non-natural amino acid residues. The terms also include post-expression modifications of the polypeptide, for example, glycosylation, sialylation, acetylation, phosphorylation, and the like. In some embodiments, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified. Amino acid analogs refer to compounds that have the same basic chemical structure as a naturally occurring amino acid. Such analogs have modified R groups or modified peptide backbones, but retain the same basic chemical structure as a naturally occurring amino acid. Amino acid mimetics refer to chemical compounds that have a structure that is different from the general chemical structure of an amino acid, but that function in a manner similar to a naturally occurring amino acid.

The term “acidic residue” refers to amino acid residues in D- or L-form having sidechains comprising acidic groups. Exemplary acidic residues include D and E.

The term “amide residue” refers to amino acid residues in D- or L-form having sidechains comprising amide derivatives of acidic groups. Exemplary amide residues include N and Q.

The term “aromatic residue” refers to amino acid residues in D- or L-form having sidechains comprising aromatic groups. Exemplary aromatic residues include F, Y, and W.

The term “basic residue” refers to amino acid residues in D- or L-form having sidechains comprising basic groups. Exemplary basic residues include H, K, and R.

The term “hydrophilic residue” refers to amino acid residues in D- or L-form having sidechains comprising polar groups. Exemplary hydrophilic residues include C, S, T, N, and Q.

The term “nonfunctional residue” refers to amino acid residues in D- or L-form having sidechains that lack acidic, basic, or aromatic groups. Exemplary nonfunctional amino acid residues include M, G, A, V, I, L and nor leucine (Nle).

The term “neutral hydrophobic residue” refers to amino acid residues in D- or L-form having sidechains that lack basic, acidic, or polar groups. Exemplary neutral hydrophobic amino acid residues include A, V, L, I, P, W, M, and F.

The term “polar hydrophobic residue” refers to amino acid residues in D- or L-form having sidechains comprising polar groups. Exemplary polar hydrophobic amino acid residues include T, G, S, Y, C, Q, and N.

The term “hydrophobic residue” refers to amino acid residues in D- or L-form having sidechains that lack basic or acidic groups. Exemplary hydrophobic amino acid residues include A, V, L, I, P, W, M, F, T, G, S, Y, C, Q, and N.

A “conservative substitution” refers to amino acid substitutions that do not significantly affect or alter binding characteristics of a particular protein. Generally, conservative substitutions are ones in which a substituted amino acid residue is replaced with an amino acid residue having a similar sidechain. Conservative substitutions include a substitution found in one of the following groups: Group 1: Alanine (Ala or A), Glycine (Gly or G), Serine (Ser or S), Threonine (Thr or T); Group 2: Aspartic acid (Asp or D), Glutamic acid (Glu or Z); Group 3: Asparagine (Asn or N), Glutamine (Gln or Q); Group 4: Arginine (Arg or R), Lysine (Lys or K), Histidine (His or H); Group 5: Isoleucine (Ile or I), Leucine (Leu or L), Methionine (Met or M), Valine (Val or V); and Group 6: Phenylalanine (Phe or F), Tyrosine (Tyr or Y), Tryptophan (Trp or W). Additionally, or alternatively, amino acids can be grouped into conservative substitution groups by similar function, chemical structure, or composition (e.g., acidic, basic, aliphatic, aromatic, or sulfur-containing). For example, an aliphatic grouping may include, for purposes of substitution, Gly, Ala, Val, Leu, and Ile. Other conservative substitutions groups include: sulfur-containing: Met and Cysteine (Cys or C); acidic: Asp, Glu, Asn, and Gin; small aliphatic, nonpolar, or slightly polar residues: Ala, Ser, Thr, Pro, and Gly; polar, negatively charged residues and their amides: Asp, Asn, Glu, and Gin; polar, positively charged residues: His, Arg, and Lys; large aliphatic, nonpolar residues: Met, Leu, Ile, Val, and Cys; and large aromatic residues: Phe, Tyr, and Trp. Additional information can be found in Creighton (1984) Proteins, W. H. Freeman and Company. Variant proteins, peptides, polypeptides, and amino acid sequences of the present disclosure can, in certain embodiments, comprise one or more conservative substitutions relative to a reference amino acid sequence.

“Nucleic acid molecule” or “polynucleotide” refers to a polymeric compound including covalently linked nucleotides comprising natural subunits (e.g., purine or pyrimidine bases). Purine bases include adenine, guanine, and pyrimidine bases including uracil, thymine, and cytosine. Nucleic acid molecules include polyribonucleic acid (RNA) and polydeoxyribonucleic acid (DNA), which includes cDNA, genomic DNA, and synthetic DNA, either of which may be single- or double-stranded. A nucleic acid molecule encoding an amino acid sequence includes all nucleotide sequences that encode the same amino acid sequence.

As used herein, the terms “homologous,” “homology,” or “percent homology” when used herein to describe to a nucleic acid sequence, relative to a reference sequence, can be determined using the formula described by Karlin & Altschul 1990, modified as in Karlin & Altschul 1993. Percent homology of sequences can be determined using the most recent version of BLAST, as of the filing date of this application.

“Percent (%) sequence identity” with respect to a reference polypeptide sequence is the percentage of amino acid residues in a candidate sequence that is identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known, for instance, using publicly available computer software such as BLAST, BLAST-2, ALIGN, or Megalign (DNASTAR) software, or other software appropriate for nucleic acid sequences. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington, D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a some % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

As used herein, “mutation” refers to a change in the sequence of a polynucleotide molecule or polypeptide molecule as compared to a reference or wild-type polynucleotide molecule or polypeptide molecule, respectively. A mutation can result in several different types of change in sequence, including substitution, insertion, or deletion of nucleotide(s) or amino acid(s).

The term “isolated” means that the material is removed from its original environment (e.g., the natural environment if it is naturally occurring). Such nucleic acid could be part of a vector and/or such nucleic acid or polypeptide could be part of a composition (e.g., a cell lysate), and still be isolated in that such vector or composition is not part of the natural environment for the nucleic acid or polypeptide.

“Exogenous” with respect to a nucleic acid or polynucleotide indicates that the nucleic acid is part of a recombinant nucleic acid construct or is not in its natural environment. For example, an exogenous nucleic acid can be a sequence from one species introduced into another species, i.e., a heterologous nucleic acid. Typically, such an exogenous nucleic acid is introduced into the other species via a recombinant nucleic acid construct. An exogenous nucleic acid also can be a sequence that is native to an organism and that has been reintroduced into cells of that organism. An exogenous nucleic acid that includes a native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic acids typically are integrated at positions other than the position where the native sequence is found. The exogenous elements may be added to a construct, for example, using genetic recombination. Genetic recombination is the breaking and rejoining of DNA strands to form new molecules of DNA encoding a novel set of genetic information.

A “functional variant” refers to a polypeptide or polynucleotide that is structurally similar or substantially structurally similar to a parent or reference compound of this disclosure, but differs, in some contexts slightly, in composition (e.g., one base, atom, or functional group is different, added, or removed; or one or more amino acids are mutated, inserted, or deleted), such that the polypeptide or encoded polypeptide is capable of performing at least one function of the encoded parent polypeptide with at least 50% efficiency of activity of the parent polypeptide.

As used herein, a “functional portion” or “functional fragment” refers to a polypeptide or polynucleotide that comprises only a domain, motif, portion, or fragment of a parent or reference compound, and the polypeptide or encoded polypeptide retains at least 50% activity associated with the domain, portion, or fragment of the parent or reference compound. In certain embodiments, a functional portion refers to a “signaling portion” of an effector molecule, effector domain, costimulatory molecule, or costimulatory domain.

The term “expression,” as used herein, refers to the process by which a polypeptide is produced based on the encoding sequence of a nucleic acid molecule, such as a gene. The process may include transcription, post-transcriptional control, post-transcriptional modification, translation, post-translational control, post-translational modification, or any combination thereof. An expressed nucleic acid molecule is typically operably linked to an expression control sequence (e.g., a promoter).

The term “operably linked” refers to the association of two or more nucleic acid molecules on a single nucleic acid fragment so that the function of one is affected by the other.

As used herein, “expression vector” refers to a DNA construct containing a nucleic acid molecule that is operably linked to a suitable control sequence capable of effecting the expression of the nucleic acid molecule in a suitable host. Such control sequences include a promoter to effect transcription, an optional operator sequence to control such transcription, a sequence encoding suitable mRNA ribosome binding sites, and sequences which control termination of transcription and translation. The vector may be a plasmid, a phage particle, a virus, or simply a potential genomic insert. Once transformed into a suitable host, the vector may replicate and function independently of the host genome, or may, in some instances, integrate into the genome itself. Here, “plasmid,” “expression plasmid,” “virus,” and “vector” are often used interchangeably.

The term “introduced,” in the context of inserting a nucleic acid molecule into a cell, means “transfection” or “transformation” or “transduction” and includes reference to the incorporation of a nucleic acid molecule into a eukaryotic cell wherein the nucleic acid molecule may be incorporated into the genome of a cell and converted into an autonomous replicon. As used herein, the term “engineered,” “recombinant” or “non-natural” refers to an organism, microorganism, cell, nucleic acid molecule, or vector that includes at least one genetic alteration or has been modified by introduction of an exogenous nucleic acid molecule, wherein such alterations or modifications are introduced by genetic engineering. Genetic alterations include, for example, modifications introducing expressible nucleic acid molecules encoding proteins, CARs, or enzymes, or other nucleic acid molecule additions, deletions, substitutions, or other functional disruption of a cell's genetic material.

The term “construct” refers to any polynucleotide that contains a recombinant nucleic acid molecule. A construct may be present in a vector (e.g., a bacterial vector, a viral vector) or may be integrated into a genome. A “vector” is a nucleic acid molecule that is capable of transporting another nucleic acid molecule. Vectors may be, for example, plasmids, cosmids, viruses, an RNA vector, or a linear or circular DNA or RNA molecule that may include chromosomal, non-chromosomal, semi-synthetic, or synthetic nucleic acid molecules. Exemplary vectors are those capable of autonomous replication (episomal vector), capable of delivering a polynucleotide to a cell genome (e.g., viral vector), or capable of expressing nucleic acid molecules to which they are linked (expression vectors).

As used herein, the term “host” refers to a cell or microorganism targeted for genetic modification with a heterologous nucleic acid molecule to produce a polypeptide of interest. In certain embodiments, a host cell may optionally already possess or be modified to include other genetic modifications that confer desired properties related or unrelated to biosynthesis of the heterologous protein.

As used herein, “enriched” or “depleted” with respect to amounts of cell types in a mixture refers to an increase in the number of the “enriched” type, a decrease in the number of the “depleted” cells, or both, in a mixture of cells resulting from one or more enriching or depleting processes or steps. In certain embodiments, amounts of a certain cell type in a mixture will be enriched and amounts of a different cell type will be depleted, such as enriching for CD4⁺ cells while depleting CD8⁺ cells, or enriching for CD8⁺ cells while depleting CD4⁺ cells, or combinations thereof.

“Chimeric antigen receptor” (CAR) refers to a CAR of the present disclosure engineered to contain two or more naturally occurring (or engineered) amino acid sequences linked together in a way that does not occur naturally or does not occur naturally in a host cell, which CAR can function as a receptor when present on a surface of a cell. CARs of the present disclosure include an extracellular portion comprising an antigen-binding domain, such as one obtained or derived from an immunoglobulin, such as an scFv derived from an antibody linked to a transmembrane region and one or more intracellular signaling domains (optionally containing co-stimulatory domain(s)) (see, e.g., Sadelain et al., 2013; see also Harris & Kranz, 2016; Stone et al., 2014).

The term “variable region” or “variable domain” refers to an antibody heavy or light chain that is involved in binding to antigen. Variable domains of antibody heavy (V_{H}) and light (V_{L}) chains each generally comprise four generally conserved framework regions (FRs) and three CDRs. Framework regions separate CDRs and CDRs are situated between framework regions.

The terms “complementarity determining region” and “CDR” are synonymous with “hypervariable region” or “HVR,” and are known in the art to refer to sequences of amino acids within antibody variable regions, which, in general, confer antigen specificity and/or binding affinity and are separated from one another in primary structure by framework sequence. In some cases, framework amino acids can also contribute to binding. In general, there are three CDRs in each variable region. Variable domain sequences can be aligned to a numbering scheme (e.g., Kabat, EU, International Immunogenetics Information System (IMGT) and Aho), which can allow equivalent residue positions to be annotated and for different molecules to be compared using Antigen receptor Numbering And Receptor Classification (ANARCI) software tool (2016, Bioinformatics 15:298-300).

“Antigen” as used herein refers to an immunogenic molecule that provokes an immune response. This immune response may involve antibody production, activation of specific immunologically competent cells, or both. An antigen may be, for example, a peptide, glycopeptide, polypeptide, glycopolypeptide, polynucleotide, polysaccharide, lipid, or the like. It is readily apparent that an antigen can be synthesized, produced recombinantly, or derived from a biological sample. Exemplary biological samples that can contain one or more antigens include tissue samples, tumor samples, cells, biological fluids, or combinations thereof. Antigens can be produced by cells that have been modified or genetically engineered to express an antigen. In some aspects, the antigen is mesothelin and/or a portion thereof.

The term “epitope” includes any molecule, structure, amino acid sequence, or protein determinant that is recognized and specifically bound by a cognate binding molecule, such as a CAR, or other binding molecule, domain, or protein.

A “binding domain” (also referred to as a “binding region”), as used herein, refers to a molecule or portion thereof that possesses the ability to specifically and non-covalently associate, unite, or combine with a target, such as an scFv. A binding domain includes any naturally occurring, synthetic, semi-synthetic, or recombinantly produced binding partner for a biological molecule, a molecular complex, or other target of interest. Exemplary binding domains include single chain immunoglobulin variable regions, receptor ectodomains, ligands, or synthetic polypeptides selected for their specific ability to bind to a biological molecule, a molecular complex, or other target of interest.

As used herein, an “effector domain” is an intracellular portion or domain of a CAR or receptor that can directly or indirectly promote a biological or physiological response in a cell when receiving an appropriate signal. In certain embodiments, an effector domain is from a protein or portion thereof or protein complex that receives a signal when bound to a target or cognate molecule, or when the protein or portion thereof or protein complex binds directly to a target or cognate molecule and triggers a signal from the effector domain.

A “transmembrane region,” as used herein, is a portion of a transmembrane protein that can insert into or span a cell membrane.

A “therapeutically effective amount,” as used herein, is an amount that produces a desired effect in a subject for treating cancer. In certain embodiments, the therapeutically effective amount is an amount that yields maximum therapeutic effect. In other embodiments, the therapeutically effective amount yields a therapeutic effect that is less than the maximum therapeutic effect. For example, a therapeutically effective amount may be an amount that produces a therapeutic effect while avoiding one or more side effects associated with a dosage that yields maximum therapeutic effect. A therapeutically effective amount for a particular composition will vary based on a variety of factors, including, but not limited to, the characteristics of the therapeutic composition (e.g., activity, pharmacokinetics, pharmacodynamics, and bioavailability), the physiological condition of the subject (e.g., age, body weight, sex, disease type and stage, medical history, general physical condition, responsiveness to a given dosage, and other present medications), the nature of any pharmaceutically acceptable carriers, excipients, and preservatives in the composition, and the route of administration. One skilled in the clinical and pharmacological arts will be able to determine a therapeutically effective amount through routine experimentation, namely, by monitoring a subject's response to administration of the therapeutic composition and adjusting the dosage accordingly. For additional guidance, see, for example, Remington: The Science and Practice of Pharmacy, 22^{nd} Edition, Pharmaceutical Press, London, 2012, and Goodman & Gilman's The Pharmacological Basis of Therapeutics, 12th Edition, McGraw-Hill, New York, NY, 2011, the entire disclosures of which are incorporated by reference herein.

The term “pharmaceutically acceptable excipient or carrier” or “physiologically acceptable excipient or carrier” refers to biologically compatible vehicles, which are described in greater detail herein, that are suitable for administration to a human or other non-human mammalian subject and generally recognized as safe or not causing a serious adverse event.

As used herein, the term “adoptive immune therapy” or “adoptive immunotherapy” refers to administration of naturally occurring or genetically engineered, disease-antigen-specific immune cells, such as T cells. Adoptive cellular immunotherapy may be autologous (immune cells are from the recipient), allogeneic (immune cells are from a donor of the same species), or syngeneic (immune cells are from a donor genetically identical to the recipient).

A “T cell” or “T lymphocyte” is an immune system cell that matures in the thymus and produces T cell receptors (TCRs), including aβT cells and γδT cells. T cells can be naïve (not exposed to antigen; increased expression of CD62L, CCR7, CD28, CD3, CD127, and CD45RA, and decreased expression of CD45RO as compared to T_{CM}), memory T cells (T_{M}) (antigen-experienced and long-lived), and effector cells (antigen-experienced, cytotoxic). T_{M} can be further divided into subsets of central memory T cells (Tan, increased expression of CD62L, CCR7, CD28, CD127, CD45RO, and CD95, and decreased expression of CD54RA as compared to naïve T cells) and effector memory T cells (TEM, decreased expression of CD62L, CCR7, CD28, CD45RA, and increased expression of CD127 as compared to naïve T cells or T_{CM}).

The term “natural killer cells” (“NK cells”), as used herein, refers to cells which are activated in response to interferons or macrophage-derived cytokines, contain viral infections while the adaptive immune response is generating antigen-specific cytotoxic T cells that can clear the infection, and express CD56.

In addition, it should be understood that the individual constructs, or groups of constructs, derived from the various combinations of the structures and subunits described herein, are disclosed by the present disclosure to the same extent as if each construct or group of constructs was set forth individually. Thus, selection of particular structures or particular subunits is within the scope of the present disclosure.

The terminology used in the description is intended to be interpreted in its broadest reasonable manner, even though it is being used in conjunction with a detailed description of identified embodiments.

### Chimeric Antigen Receptors (CARs)

Aspects of the present disclosure are directed to chimeric antigen receptors (CAR) which bind to at least a portion of mesothelin that is expressed at least partially on an extracellular surface of a cell, such as a malignant cell, or shed by the same.

In certain aspects, the present disclosure provides CARs comprising (a) an extracellular region comprising a binding domain that specifically binds to at least a portion of mesothelin, (b) a transmembrane region, and (c) an intracellular region comprising an effector domain or a portion or variant thereof and a costimulatory domain or a portion or variant thereof.

Mesothelin binding domains disposed in the (a) extracellular regions of the present disclosure are, in some embodiments, scFvs which comprise at least a portion of an antibody V_{L} chain, at least a portion of an antibody V_{H} chain, and a linker domain. In some embodiments, the at least a portion of the antibody V_{L} chain is a V_{L} domain and the at least a portion of the V_{H} chain is the V_{H} domain. In some embodiments, the linker domain is a peptide linker disposed between the V_{L} domain and the V_{H} domain. For example, the mesothelin scFvs may be designed so that the C-terminal end of the V_{L} domain is linked to the N-terminal end of the V_{H} domain by the peptide linker ((N)V_{L}(C)-linker-(N)V_{H}(C)) or such that the C-terminal end of the V_{H} domain is linked to the N-terminal end of the V_{L} domain by the peptide linker (N)V_{H}(C)-linker-(N)V_{L}(C). Exemplary linkers include those having a glycine-serine amino acid chain having from one to about ten repeats of GlyₓSer_{y}, wherein x and y are each independently an integer from 0 to 10, provided that x and y are not both 0 (e.g., (Gly₄Ser)₂; (Gly₃Ser)₂; Gly₂Ser; or a combination thereof, such as (Gly₃Ser)₂Gly₂Ser). Linker length may be varied to maximize mesothelin antigen recognition based on mesothelin, the selected binding epitope of mesothelin, or mesothelin antigen binding domain size and affinity.

Sources of binding domains specific for mesothelin are known in the art, including known antibodies, methods of generating mesothelin antibodies, and mesothelin binding domains described herein. Exemplary binding domains specific for mesothelin antigens, including CDRs thereof, are disclosed at SEQ ID NOs: 1 and 3. In certain embodiments, the mesothelin scFV portion or variant thereof comprises or consists of an amino acid sequence having at least 75%, 80%, 85%, 90%, 91%, 92%, 93%, 94%, 95%, 96%, 97%, 98%, 99%, or 100% identity to any one of SEQ ID NOs: 1-3.

Mesothelin CARs of the present disclosure comprise one or more CDRs, such as three heavy chain CDRs and three light chain CDRs, according to any one of these exemplary binding domain SEQ ID NOs: 1 and 3 or can comprise a portion or a variant sequence thereof. Mesothelin binding domain affinities can be determined using a variety of known assays, such as Western blot, ELISA, analytical ultracentrifugation, spectroscopy and surface plasmon resonance (Biacore®) analysis (see, e.g., Scatchard et al, Ann. N.Y. Acad. Sci. 51:660 (1949); Wilson 2002; Wolff et al., 1993; and U.S. Pat. Nos. 5,283,173, 5,468,614, or the equivalent).

In some embodiments, the (a) extracellular region further comprises a leader domain, which includes but is not limited to, a leader peptide and/or a signal peptide. For example, the signal peptide can be a GM-CSFR signal peptide, or a portion or variant thereof bound to the N-terminal end of the V_{H} domain or the V_{L} domain of the mesothelin scFv. An exemplary signal peptide is disclosed at SEQ ID NO: 4.

In some embodiments, the (c) intracellular region effector domain is from CD3 or a functional portion or variant thereof. An exemplary CD3 effector domain is disclosed at SEQ ID NO: 5. In certain embodiments, the CD3 effector domain portion or variant thereof comprises or consists of an amino acid sequence having at least 75%, 80%, 85%, 90%, 91%, 92%, 93%, 94%, 95%, 96%, 97%, 98%, 99%, or 100% identity to SEQ ID NO: 5.

In some embodiments, the (c) intracellular region costimulatory domain is an intracellular tail from 4-1BB or portion or variant thereof. The costimulatory domain is disposed between the transmembrane domain or portion or variant thereof and the effector domain portion or variant thereof. In certain embodiments, the intracellular tail from 4-1BB or portion or variant thereof comprises or consists of an amino acid sequence having at least 75%, 80%, 85%, 90%, 91%, 92%, 93%, 94%, 95%, 96%, 97%, 98%, 99%, or 100% identity to SEQ ID NO: 10.

The (a) extracellular region and the (c) intracellular region of the present disclosure are connected by the (b) transmembrane region. For example, the (b) transmembrane region is disposed between a C-terminal end of the mesothelin scFv and an N-terminal end of the (c) intracellular region. In certain embodiments, the transmembrane region comprises or is derived from a known transmembrane protein, such as a CD28 transmembrane region. In some embodiments, the (b) transmembrane region comprises a known hinge domain and a known transmembrane domain, such as an IgG4 hinge domain and the transmembrane domain of CD28, or a portion or variant thereof. An exemplary IgG4 hinge domain and CD28 transmembrane domain are disclosed at SEQ ID NOs: 6 and 9, respectively. In certain embodiments, the IgG4 hinge domain and CD28 transmembrane domain portion or variant thereof comprises or consists of an amino acid sequence having at least 75%, 80%, 85%, 90%, 91%, 92%, 93%, 94%, 95%, 96%, 97%, 98%, 99% or 100% identity to SEQ ID NOs: 6 and 9, respectively.

In some embodiments, there may be an additional spacer of various length in between the hinge domain and the transmembrane domain. Exemplary spacers are disclosed at SEQ ID NOs: 7-8. In certain embodiments, the spacer comprises or consists of an amino acid sequence having at least 75%, 80%, 85%, 90%, 91%, 92%, 93%, 94%, 95%, 96%, 97%, 98%, 99% or 100% identity to SEQ ID NO: 7 or 8.

Polypeptide markers are unique peptide sequences that are co-expressed in a cell, such as a host cell, along with one or more mesothelin CARs. Unlike mesothelin CARs of the present disclosure, polypeptide markers are recognized or bound by, for example, an antibody. Polypeptide markers can be useful for detecting, identifying, isolating, tracking, purifying, enriching for, targeting, or biologically or chemically modifying tagged proteins of interest, particularly when a tagged protein is part of a heterogeneous population of cell proteins or cells, such as a biological sample like peripheral blood. Exemplary polypeptide markers of the present disclosure include a truncated form of CD19 (CD19t). An exemplary CD19t amino acid sequence is shown as SEQ ID NO: 12. In some embodiments, the amino acid sequence for CD19t is separated from the mesothelin CAR by a T2A sequence, such as that shown in SEQ ID NO: 11.

TABLE 1
Mesothelin CAR Amino Acid Sequences

| SEQ ID | | |
|---|---|---|
| NO: | NAME | SEQUENCE |
| 1 | V_{L} of SS1 | DIELTQSPAIMSASPGEKVTMTCSASSSVSYMHWYQQKSGTSPKRWIYD |
| | | TSKLASGVPGRFSGSGSGNSYSLTISSVEAEDDATYYCQQWSGYPLTFG |
| | | AGTKLEIK |
| 2 | (G₄S)₄ | GGGGSGGGGSGGGGSGGGGS |
| 3 | V_{H} of SS1 | QVQLQQSGPELEKPGASVKISCKASGYSFTGYTMNWVKQSHGKSLEWIG |
| | | LITPYNGASSYNQKFRGKATLTVDKSSSTAYMDLLSLTSEDSAVYFCAR |
| | | GGYDGRGFDYWGQGTTVTVSS |
| 4 | GM-CSFR | MLLLVTSLLLCELPHPAFLLIP |
| | signal | |
| | peptide | |
| 5 | CD3ζ | RVKFSRSADAPAYQQGQNQLYNELNLGRREEYDVLDKRRGRDPEMGGKP |
| | effector | RRKNPQEGLYNELQKDKMAEAYSEIGMKGERRRGKGHDGLYQGLSTATK |
| | domain | DTYDALHMQALPPR |
| 6 | IgG4 hinge | ESKYGP |
| | domain | |
| 7 | Intermediate | PCPPCPGQPREPQVYTLPPSQEEMTKNQVSLTCLVKGFYPSDIAVEWES |
| | spacer | NGQPENNYKTTPPVLDSDGSFFLYSRLTVDKSRWQEGNVFSCSVMHEAL |
| | | HNHYTQKSLSLSLGK |
| 8 | Long spacer | PCPPCPAPPVAGPSVFLFPPKPKDTLMISRTPEVTCVVVDVSQEDPEVQ |
| | | FNWYVDGVEVHNAKTKPREEQFQSTYRVVSVLTVLHQDWLNGKEYKCKV |
| | | SNKGLPSSIEKTISKAKGQPREPQVYTLPPSQEEMTKNQVSLTCLVKGF |
| | | YPSDIAVEWESNGQPENNYKTTPPVLDSDGSFFLYSRLTVDKSRWQEGN |
| | | VFSCSVMHEALHNHYTQKSLSLSLGK |
| 9 | CD28 | MFWVLVVVGGVLACYSLLVTVAFIIFW |
| | transmembrane | |
| | (TM) domain | |
| 10 | 4-1BB | KRGRKKLLYIFKQPFMRPVQTTQEEDGCSCRFPEEEEGGCEL |
| | costimulatory | |
| | domain | |
| 11 | T2A | LEGGGEGRGSLLTCGDVEENPGPR |
| 12 | Truncated | MPPPRLLFFLLFLTPMEVRPEEPLVVKVEEGDNAVLQCLKGTSDGPTQQ |
| | CD19 | LTWSRESPLKPFLKLSLGLPGLGIHMRPLAIWLFIFNVSQQMGGFYLCQ |
| | (CD19t) | PGPPSEKAWQPGWTVNVEGSGELFRWNVSDLGGLGCGLKNRSSEGPSSP |
| | | SGKLMSPKLYVWAKDRPEIWEGEPPCVPPRDSLNQSLSQDLTMAPGSTL |
| | | WLSCGVPPDSVSRGPLSWTHVHPKGPKSLLSLELKDDRPARDMWVMETG |
| | | LLLPRATAQDAGKYYCHRGNLTMSFHLEITARPVLWHWLLRTGGWKVSA |
| | | VTLAYLIFCLCSLVGILHLQRALVLRRKR |
| 13 | Short | MLLLVTSLLLCELPHPAFLLIPDIELTQSPAIMSASPGEKVTMTCSASS |
| | Mesothelin | SVSYMHWYQQKSGTSPKRWIYDTSKLASGVPGRFSGSGSGNSYSLTISS |
| | CAR | VEAEDDATYYCQQWSGYPLTFGAGTKLEIKGGGGSGGGGSGGGGSGGGG |
| | | SQVQLQQSGPELEKPGASVKISCKASGYSFTGYTMNWVKQSHGKSLEWI |
| | | GLITPYNGASSYNQKFRGKATLTVDKSSSTAYMDLLSLTSEDSAVYFCA |
| | | RGGYDGRGFDYWGQGTTVTVSSESKYGPMFWVLVVVGGVLACYSLLVTV |
| | | AFIIFWKRGRKKLLYIFKQPFMRPVQTTQEEDGCSCRFPEEEEGGCELR |
| | | VKFSRSADAPAYQQGQNQLYNELNLGRREEYDVLDKRRGRDPEMGGKPR |
| | | RKNPQEGLYNELQKDKMAEAYSEIGMKGERRRGKGHDGLYQGLSTATKD |
| | | TYDALHMQALPPRLEGGGEGRGSLLTCGDVEENPGPRMPPPRLLFFLLF |
| | | LTPMEVRPEEPLVVKVEEGDNAVLQCLKGTSDGPTQQLTWSRESPLKPF |
| | | LKLSLGLPGLGIHMRPLAIWLFIFNVSQQMGGFYLCQPGPPSEKAWQPG |
| | | WTVNVEGSGELFRWNVSDLGGLGCGLKNRSSEGPSSPSGKLMSPKLYVW |
| | | AKDRPEIWEGEPPCVPPRDSLNQSLSQDLTMAPGSTLWLSCGVPPDSVS |
| | | RGPLSWTHVHPKGPKSLLSLELKDDRPARDMWVMETGLLLPRATAQDAG |
| | | KYYCHRGNLTMSFHLEITARPVLWHWLLRTGGWKVSAVTLAYLIFCLCS |
| | | LVGILHLQRALVLRRKR |
| 14 | Intermediate | MLLLVTSLLLCELPHPAFLLIPDIELTQSPAIMSASPGEKVTMTCSASS |
| | Mesothelin | SVSYMHWYQQKSGTSPKRWIYDTSKLASGVPGRFSGSGSGNSYSLTISS |
| | CAR | VEAEDDATYYCQQWSGYPLTFGAGTKLEIKGGGGSGGGGSGGGGSGGGG |
| | | SQVQLQQSGPELEKPGASVKISCKASGYSFTGYTMNWVKQSHGKSLEWI |
| | | GLITPYNGASSYNQKFRGKATLTVDKSSSTAYMDLLSLTSEDSAVYFCA |
| | | RGGYDGRGFDYWGQGTTVTVSSESKYGPPCPPCPGQPREPQVYTLPPSQ |
| | | EEMTKNQVSLTCLVKGFYPSDIAVEWESNGQPENNYKTTPPVLDSDGSF |
| | | FLYSRLTVDKSRWQEGNVFSCSVMHEALHNHYTQKSLSLSLGKMFWVLV |
| | | VVGGVLACYSLLVTVAFIIFWKRGRKKLLYIFKQPFMRPVQTTQEEDGC |
| | | SCRFPEEEEGGCELRVKFSRSADAPAYQQGQNQLYNELNLGRREEYDVL |
| | | DKRRGRDPEMGGKPRRKNPQEGLYNELQKDKMAEAYSEIGMKGERRRGK |
| | | GHDGLYQGLSTATKDTYDALHMQALPPRLEGGGEGRGSLLTCGDVEENP |
| | | GPRMPPPRLLFFLLFLTPMEVRPEEPLVVKVEEGDNAVLQCLKGTSDGP |
| | | TQQLTWSRESPLKPFLKLSLGLPGLGIHMRPLAIWLFIFNVSQQMGGFY |
| | | LCQPGPPSEKAWQPGWTVNVEGSGELFRWNVSDLGGLGCGLKNRSSEGP |
| | | SSPSGKLMSPKLYVWAKDRPEIWEGEPPCVPPRDSLNQSLSQDLTMAPG |
| | | STLWLSCGVPPDSVSRGPLSWTHVHPKGPKSLLSLELKDDRPARDMWVM |
| | | ETGLLLPRATAQDAGKYYCHRGNLTMSFHLEITARPVLWHWLLRTGGWK |
| | | VSAVTLAYLIFCLCSLVGILHLQRALVLRRKR |
| 15 | Long | MLLLVTSLLLCELPHPAFLLIPDIELTQSPAIMSASPGEKVTMTCSASS |
| | Mesothelin | SVSYMHWYQQKSGTSPKRWIYDTSKLASGVPGRFSGSGSGNSYSLTISS |
| | CAR | VEAEDDATYYCQQWSGYPLTFGAGTKLEIKGGGGSGGGGSGGGGSGGGG |
| | | SQVQLQQSGPELEKPGASVKISCKASGYSFTGYTMNWVKQSHGKSLEWI |
| | | GLITPYNGASSYNQKFRGKATLTVDKSSSTAYMDLLSLTSEDSAVYFCA |
| | | RGGYDGRGFDYWGQGTTVTVSSESKYGPPCPPCPAPPVAGPSVFLFPPK |
| | | PKDTLMISRTPEVTCVVVDVSQEDPEVQFNWYVDGVEVHNAKTKPREEQ |
| | | FQSTYRVVSVLTVLHQDWLNGKEYKCKVSNKGLPSSIEKTISKAKGQPR |
| | | EPQVYTLPPSQEEMTKNQVSLTCLVKGFYPSDIAVEWESNGQPENNYKT |
| | | TPPVLDSDGSFFLYSRLTVDKSRWQEGNVFSCSVMHEALHNHYTQKSLS |
| | | LSLGKMFWVLVVVGGVLACYSLLVTVAFIIFWKRGRKKLLYIFKQPFMR |
| | | PVQTTQEEDGCSCRFPEEEEGGCELRVKFSRSADAPAYQQGQNQLYNEL |
| | | NLGRREEYDVLDKRRGRDPEMGGKPRRKNPQEGLYNELQKDKMAEAYSE |
| | | IGMKGERRRGKGHDGLYQGLSTATKDTYDALHMQALPPRLEGGGEGRGS |
| | | LLTCGDVEENPGPRMPPPRLLFFLLFLTPMEVRPEEPLVVKVEEGDNAV |
| | | LQCLKGTSDGPTQQLTWSRESPLKPFLKLSLGLPGLGIHMRPLAIWLFI |
| | | FNVSQQMGGFYLCQPGPPSEKAWQPGWTVNVEGSGELFRWNVSDLGGLG |
| | | CGLKNRSSEGPSSPSGKLMSPKLYVWAKDRPEIWEGEPPCVPPRDSLNQ |
| | | SLSQDLTMAPGSTLWLSCGVPPDSVSRGPLSWTHVHPKGPKSLLSLELK |
| | | DDRPARDMWVMETGLLLPRATAQDAGKYYCHRGNLTMSFHLEITARPVL |
| | | WHWLLRTGGWKVSAVTLAYLIFCLCSLVGILHLQRALVLRRKR |

### Polynucleotides, Vectors, and Host Cells

Nucleic acid molecules and polynucleotides are provided that encode any one or more of the mesothelin CARs or variants or portions thereof as described herein. In any of the embodiments described herein, mesothelin CARs, portions, or variants thereof may be codon-optimized for a host cell containing the polynucleotide using known techniques (Scholten et al., 2006). Codon optimization can be performed using, e.g., the GenScript® OptimumGene™ tool. Codon-optimized sequences include sequences that are partially or fully codon-optimized.

A polynucleotide encoding a mesothelin CAR of this disclosure can be inserted into an expression vector, such as a viral vector, for transduction into a host cell, such as a T cell. In some embodiments, an expression construct of the present disclosure comprises a mesothelin CAR polynucleotide and optionally a T2A self-cleaving peptide (e.g., SEQ ID NO. 11) and optionally a CD19t marker (e.g., SEQ ID NO. 12) operably linked to an expression control sequence such as a promoter.

In certain embodiments, polynucleotides of the present disclosure may be operatively linked to certain elements of the vector. For example, polynucleotide sequences that are needed to affect the expression and processing of coding sequences to which they are ligated may be operatively linked. Expression control sequences may include appropriate transcription initiation, termination, promoter and enhancer sequences; efficient RNA processing signals such as splicing and polyadenylation signals; sequences that stabilize cytoplasmic mRNA; sequences that enhance translation efficiency; sequences that enhance protein stability; and possibly sequences that enhance protein secretion. Expression control sequences may be operatively linked if they are contiguous with the gene of interest and expression control sequences that act in trans or at a distance to control the gene of interest.

In certain embodiments, the expression construct is comprised in a vector which may integrate into a host cell's genome or promote integration of the polynucleotide insert upon introduction into the host cell and thereby replicate along with the host genome, such as a viral vector. Viral vectors include retrovirus, adenovirus, parvovirus, coronavirus, negative strand RNA viruses, positive strand RNA viruses, and double-stranded DNA viruses. (Coffin, J. M., Retroviridae: The viruses and their replication, In Fundamental Virology, Third Edition, B. N. Fields et al., Eds., Lippincott-Raven Publishers, Philadelphia, 1996).

Construction of an expression vector that is used for genetically engineering and producing a CAR of interest can be accomplished by using any suitable molecular biology engineering techniques known in the art. To obtain efficient transcription and translation, a polynucleotide in each recombinant expression construct includes at least one appropriate expression control sequence, such as a leader sequence and particularly a promoter operably linked to the nucleotide sequence encoding the immunogen. Methods for making CARs of the present disclosure are described, for example, in U.S. Pat. Nos. 6,410,319; 7,446,191; U.S. Patent Publ.

No. 2010/065818; U.S. Pat. No. 8,822,647; PCT Publ. No. WO 2014/031687; U.S. Pat. No. 7,514,537; Brentjens et al., 2007; and Walseng et al., 2017; the techniques of which are herein incorporated by reference.

In certain embodiments, polynucleotides of the present disclosure are used to transfect/transduce a host cell, such as a T cell or an NK cell, for use in adoptive transfer therapy to target mesothelin. Cells may be induced to incorporate the vector or other material by use of a viral vector, transformation via calcium phosphate precipitation, DEAE-dextran, electroporation, microinjection, or other methods. (Sambrook et al., Molecular Cloning: A Laboratory Manual 2d ed. (Cold Spring Harbor Laboratory, 1989)). T cells and/or NK cells can be collected using known techniques, and the various subpopulations or combinations thereof can be enriched or depleted by known techniques, such as by affinity binding to antibodies, flow cytometry, or immunomagnetic selection. In certain embodiments, the T cell is a CD4+ T cell, a CD8+ T cell, a CD4− CD8− double negative T cell, a naïve T cell, a central memory T cell, an effector memory T cell, a stem cell memory T cell, or any combination thereof. Methods for transfecting/transducing T cells with polynucleotides have been previously described (U.S. Patent Application Pub. No. US 2004/0087025) as have adoptive transfer procedures using T cells of desired target-specificity (Schmitt et al., 2009; Dossett et al., 2009; Till et al. 2008; Wang et al., 2007; Kuball et al., 2007; Leen et al., 2007; U.S. Patent Publ. No. 2011/0243972; U.S. Patent Publ. No. 2011/0189141), such that adaptation of these methodologies to the presently disclosed mesothelin CARs of the present disclosure is within the scope of the present disclosure.

In certain embodiments, a mesothelin CAR of the instant disclosure is expressed by a host cell, such as a T cell and/or an NK cell, and the host cell recognizes and initiates an immune response to a target cell expressing mesothelin. As explained in greater detail below, the target cell includes malignant cells, such as cancer cells, and in particular, AML cells.

In any of the embodiments disclosed herein, mesothelin CARs, when expressed by a host cell such as a T cell and/or an NK cell, result in at least one of the following outcomes: (i) improved cell signaling, cytotoxic activity, proliferation, and/or survival in response to mesothelin relative to a T cell and/or an NK cell that does not express the mesothelin CAR of the present disclosure, wherein improved cell signaling optionally comprises increased and/or sustained cytokine production and/or release, and/or phosphorylation of one or more proteins associated with an immune cell response to antigen-binding, or any combination thereof; (ii) improved cell activity in response to antigen relative to a T cell and/or an NK cell that does not express the mesothelin CAR of the present disclosure, wherein improved cell signaling optionally comprises increased mobilization of intracellular calcium, killing activity, proliferation, earlier activation in response to antigen, or any combination thereof; (iii) improved cell signaling and/or activity, relative to a T cell and/or an NK cell that does not express the mesothelin CAR of the present disclosure, upon binding to a target antigen that is expressed at a low level or an intermediate level on a target cell surface; (iv) reducing or suppressing growth, area, volume, and/or spread of a tumor that expresses an antigen that is recognized and/or specifically bound by the mesothelin CAR, of killing tumor cells, and/or of increasing survival of the subject to a greater degree and/or for a longer period of time as compared to a T cell and/or an NK cell that does not express the mesothelin CAR of the present disclosure; (v) improved sensitivity to mesothelin antigen expression compared to a T cell that does not express the mesothelin CAR of the present disclosure; or (vi) any combination of (i)-(v).

Functional characterization of mesothelin CARs described herein may be performed according to any art-accepted methodologies for assaying T cell and/or NK cell activity, including determination of T cell and/or NK cell binding, activation, or induction and also including determination of T cell and/or NK cell responses that are antigen-specific. Examples include determination of intracellular calcium, T cell proliferation, T cell and/or NK cell cytokine release, antigen-specific T cell and/or NK cell stimulation, MHC-restricted T cell and/or NK cell stimulation, cytotoxic activity, changes in T cell and/or NK cell phenotypic marker expression, phosphorylation of certain T cell and/or NK cell proteins, and other measures of T cell and/or NK cell functions. Procedures for performing these and similar assays are described herein and/or may be found, for example, in Lefkovits (Immunology Methods Manual: The Comprehensive Sourcebook of Techniques, 1998). See, also, Current Protocols in Immunology; Weir, Handbook of Experimental Immunology, Blackwell Scientific, Boston, M A (1986); Mishell and Shigii (eds.), Selected Methods in Cellular Immunology, Freeman Publishing, San Francisco, C A (1979); Green and Reed, Science 281:1309 (1998) and references cited therein.

In some embodiments, kits are provided comprising (a) a mesothelin CAR vector encoding at least one of the mesothelin CARs described herein (SEQ ID NOs. 1-10 and 13-15), (b) a mesothelin CAR polynucleotide, (c) a marker peptide and self-cleaving peptide (SEQ ID NOs: 11 and 12), (d) instructions, and/or (e) one or more reagents for transducing the vector or polynucleotides into a host cell.

### Uses

The present disclosure also provides methods for treating a disease or condition, wherein the methods comprise administering to a subject in need thereof an effective amount of a host cell, composition, or unit dose of the present disclosure, wherein the disease or condition expresses or is otherwise associated with the antigen that is specifically bound by the CAR. In certain embodiments, the disease or condition is a hyperproliferative or proliferative disease, such as a cancer, and in particular, AML.

Subjects that can be treated by the present invention are, in general, human and other primate subjects, such as monkeys and apes for veterinary medicine purposes. In any of the aforementioned embodiments, the subject may be a human subject. The subjects can be male or female and can be any suitable age, including infant, juvenile, adolescent, adult, and geriatric subjects. Cells according to the present disclosure may be administered in a manner appropriate to the disease, condition, or disorder to be treated as determined by persons skilled in the medical art. In any of the above embodiments, a cell comprising a CAR as described herein is administered intravenously, intraperitoneally, intratumorally, into the bone marrow, into a lymph node, or into the cerebrospinal fluid so as to encounter the target antigen or cells. An appropriate dose, suitable duration, and frequency of administration of the compositions will be determined by such factors as a condition of the patient; size, type, and severity of the disease, condition, or disorder; the undesired type or level or activity of the tagged cells; the particular form of the active ingredient; and the method of administration.

In any of the above embodiments, methods of the present disclosure comprise administering a host cell expressing a CAR of the present disclosure, or a composition comprising the host cell. The amount of cells in a composition is at least one cell (for example, one CAR-modified CD8+ T cell subpopulation; one CAR-modified CD4+ T cell subpopulation; one CAR-modified NK cell subpopulation) or is more typically greater than 10² cells, for example, up to 10⁶ cells, up to 10⁷ cells, up to 10⁸ cells, up to 10⁹ cells, or 10¹⁰ cells or more, such as about 10¹¹ cells/m². In certain embodiments, the cells are administered in a range from about 10⁵ to about 10¹¹ cells/m², preferably in a range of about 10⁵ or about 10⁶ to about 10⁹ or about 10¹⁰ cells/m². The number of cells will depend upon the ultimate use for which the composition is intended as well as the type of cells included therein. For example, cells modified to contain a CAR specific for a particular antigen will comprise a cell population containing at least 30%, 35%, 40%, 45%, 50%, 55%, 60%, 65%, 70%, 75%, 80%, 85%, 90%, 95% or more of such cells. For uses provided herein, cells are generally in a volume of a liter or less, 500 mls or less, 250 m Is or less, or 100 mls or less. In embodiments, the density of the desired cells is typically greater than 10⁴ cells/ml and generally is greater than 10⁷ cells/m 1, generally 10⁸ cells/m 1 or greater. The cells may be administered as a single infusion or in multiple infusions over a range of time. A clinically relevant number of immune cells can be apportioned into multiple infusions that cumulatively equal or exceed 10⁶, 10⁷, 10⁸, 10⁹, 10¹⁰, or 10¹¹ cells. In any of the presently disclosed embodiments, the host cell is an allogeneic cell, a syngeneic cell, or an autologous cell.

Unit doses are also provided herein which comprise a host cell (e.g., a modified immune cell comprising a polynucleotide of the present disclosure) or host cell composition of this disclosure. In some embodiments, a unit dose comprises (i) a composition comprising at least about 50% modified CD4+ T cells, combined with (ii) a composition comprising at least about 50% modified CD8+ T cells, in about a 1:1 ratio, wherein the unit dose contains a reduced amount or substantially no naïve T cells.

Also contemplated are pharmaceutical compositions that comprise cells expressing the CARs as disclosed herein and a pharmaceutically acceptable carrier, diluents, or excipient. Suitable excipients include water, saline, dextrose, glycerol, or the like and combinations thereof. In some embodiments, compositions comprising host cells as disclosed herein further comprise a suitable infusion media.

Pharmaceutical compositions may be administered in a manner appropriate to the disease or condition to be treated (or prevented) as determined by persons skilled in the medical art. An appropriate dose and a suitable duration and frequency of administration of the compositions will be determined by such factors as the health condition of the patient, size of the patient (i.e., weight, mass, or body area), the type and severity of the patient's condition, the undesired type or level or activity of the tagged cells, the particular form of the active ingredient, and the method of administration. In general, an appropriate dose and treatment regimen provide the composition(s) in an amount sufficient to provide therapeutic and/or prophylactic benefit (such as described herein, including an improved clinical outcome, such as more frequent complete or partial remissions, or longer disease-free and/or overall survival, or a lessening of symptom severity). For prophylactic use, a dose should be sufficient to prevent, delay the onset of, or diminish the severity of a disease or disorder. Prophylactic benefit of the immunogenic compositions administered according to the methods described herein can be determined by performing pre-clinical (including in vitro and in vivo animal studies) and clinical studies and analyzing data obtained therefrom by appropriate statistical, biological, and clinical methods and techniques, all of which can readily be practiced by a person skilled in the art.

Certain methods of treatment or prevention contemplated herein include administering a host cell (which may be autologous, allogeneic, or syngeneic) comprising a desired polynucleotide as described herein that is stably integrated into the chromosome of the cell. For example, such a cellular composition may be generated ex vivo using autologous, allogeneic, or syngeneic immune system cells (e.g., T cells, antigen-presenting cells, NK cells) in order to administer a desired, CAR-expressing T-cell composition to a subject as an adoptive immunotherapy. In certain embodiments, the host cell is a hematopoietic progenitor cell or a human immune cell. In certain embodiments, the immune system cell is a CD4+ T cell, a CD8+ T cell, a CD4− CD8− double-negative T cell, an NK cell, or any combination thereof. In certain embodiments, the immune system cell is a naïve T cell, a central memory T cell, a stem cell memory T cell, an effector memory T cell, an NK cell, or any combination thereof. In particular embodiments, the cell is a CD4+ T cell. In particular embodiments, the cell is a CD8+ T cell. In particular embodiments, the cell is an NK cell.

As used herein, administration of a composition refers to delivering the same to a subject, regardless of the route or mode of delivery. Administration may be affected continuously or intermittently, and parenterally. Administration may be for treating a subject already confirmed as having a recognized condition, disease, or disease state, or for treating a subject susceptible to or at risk of developing such a condition, disease, or disease state. Co-administration with an adjunctive therapy may include simultaneous and/or sequential delivery of multiple agents in any order and on any dosing schedule (e.g., CAR-expressing recombinant (i.e., engineered) host cells with one or more cytokines; immunosuppressive therapy such as calcineurin inhibitors, corticosteroids, microtubule inhibitors, low dose of a mycophenolic acid prodrug, or any combination thereof).

In certain embodiments, a plurality of doses of a recombinant host cell as described herein is administered to the subject, which may be administered at intervals between administrations of about two to about four weeks.

In still further embodiments, the subject being treated is further receiving immunosuppressive therapy, such as calcineurin inhibitors, corticosteroids, microtubule inhibitors, low dose of a mycophenolic acid prodrug, or any combination thereof. In yet further embodiments, the subject being treated has received a non-myeloablative or a myeloablative hematopoietic cell transplant, wherein the treatment may be administered at least two to at least three months after the non-myeloablative hem atopoietic cell transplant.

An effective amount of a pharmaceutical composition (e.g., host cell, CAR, unit dose, or composition) refers to an amount sufficient, at dosages and for periods of time needed, to achieve the desired clinical results or beneficial treatment, as described herein. An effective amount may be delivered in one or more administrations.

Methods according to this disclosure may further include administering one or more additional agents to treat the disease or disorder in a combination therapy. For example, in certain embodiments, a combination therapy comprises administering a CAR (or an engineered host cell expressing the same) with (concurrently, simultaneously, or sequentially) an immune checkpoint inhibitor. In some embodiments, a combination therapy comprises administering a CAR of the present disclosure (or an engineered host cell expressing the same) with an agonist of a stimulatory immune checkpoint agent. In further embodiments, a combination therapy comprises administering a CAR of the present disclosure (or an engineered host cell expressing the same) with a secondary therapy, such as a chemotherapeutic agent, a radiation therapy, a surgery, an antibody, or any combination thereof.

Cytokines are used to manipulate host immune response towards anticancer activity (see, e.g., Floros & Tarhini, 2015). Cytokines useful for promoting immune anticancer or antitumor response include, for example, IFN-α, IL-2, IL-3, IL-4, IL-10, IL-12, IL-13, IL-15, IL-16, IL-17, IL-18, IL-21, IL-24, and GM-CSF, singly or in any combination with the binding proteins or cells expressing the same.

Various embodiments of the technology are described above. It will be appreciated that details set forth above are provided to describe the embodiments in a manner sufficient to enable a person skilled in the relevant art to make and use the disclosed embodiments. Several of the details and advantages, however, may not be necessary to practice some embodiments. Additionally, some well-known structures or functions may not be shown or described in detail, so as to avoid unnecessarily obscuring the relevant description of the various embodiments. Although some embodiments may be within the scope of the technology, they may not be described in detail with respect to the Figures. Furthermore, features, structures, or characteristics of various embodiments may be combined in any suitable manner. Moreover, one skilled in the art will recognize that there are a number of other technologies that could be used to perform functions similar to those described above. While processes or blocks are presented in a given order, alternative embodiments may perform routines having stages, or employ systems having blocks, in a different order, and some processes or blocks may be deleted, moved, added, subdivided, combined, and/or modified. Each of these processes or blocks may be implemented in a variety of different ways. Also, while processes or blocks are at times shown as being performed in series, these processes or blocks may instead be performed in parallel or may be performed at different times. The headings provided herein are for convenience only and do not interpret the scope or meaning of the described technology.

These and other changes can be made in light of the above Detailed Description. While the above description details certain embodiments and describes the best mode contemplated, no matter how detailed, various changes can be made. Implementation details may vary considerably, while still being encompassed by the technology disclosed herein. As noted above, particular terminology used when describing certain features or aspects of the technology should not be taken to imply that the terminology is being redefined herein to be restricted to any specific characteristics, features, or aspects of the technology with which that terminology is associated.

The foregoing is merely intended to illustrate various embodiments of the present invention. The specific modifications discussed above are not to be construed as limitations on the scope of the invention. It will be apparent to one skilled in the art that various equivalents, changes, and modifications may be made without departing from the scope of the invention, and it is understood that such equivalent embodiments are to be included herein. All references cited herein are incorporated by reference as if fully set forth herein.

The following example is illustrative of several embodiments of the present technology.

### Example

Mesothelin is actively cleaved at the cell membrane (shedding) contributing to an antigen pool of soluble mesothelin in the blood circulation which can be used for detection and diagnosis but may hinder antibody-based therapies¹⁻⁵. Without intending to be bound by any particular theory, it is thought that the shed form can limit CAR T functionality by competing for the antigen binding domain of the CAR resulting in immune escape^{1,6-9}. Shedding may also reduce antigen site density and may promote the dissociation of CAR bound to mesothelin from the cell surface after binding², which may further ham per the ability of CAR T cells to recognize tumor cells. ADAM17 (TACE) is a well-known “sheddase” that is responsible for the release of many membrane-bound proteins including cytokines, adhesion molecules, receptors, ligands and enzymes (Review in¹⁰) and it is implicated in drug resistance^{11,12}. Mesothelin is a target of ADAM17 cleavage, and, without intending to be bound by any particular theory, it is thought that inhibition of ADAM17 with the pan-metalloproteinase inhibitor GM6001^{3,5}, or by mutating the ADAM17 cleavage site¹, leads to increased cell surface density of mesothelin, decreased production of soluble mesothelin, and enhanced cytotoxicity of anti-mesothelin targeted therapies.

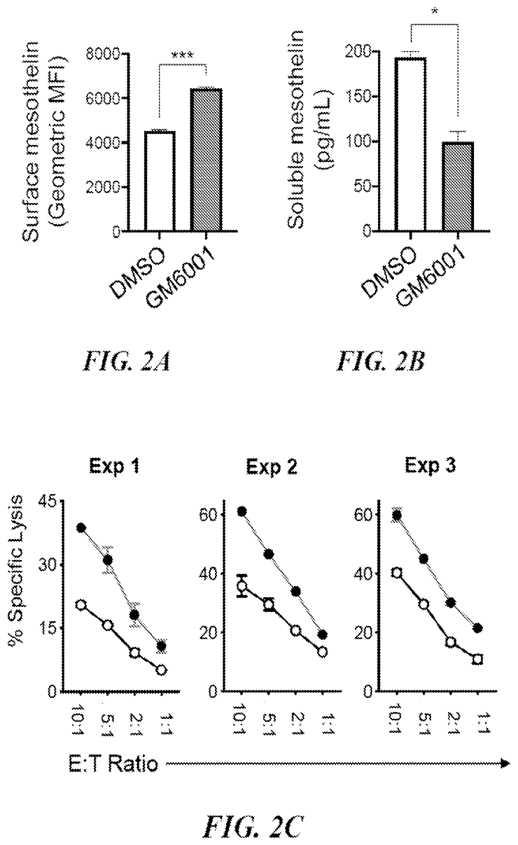

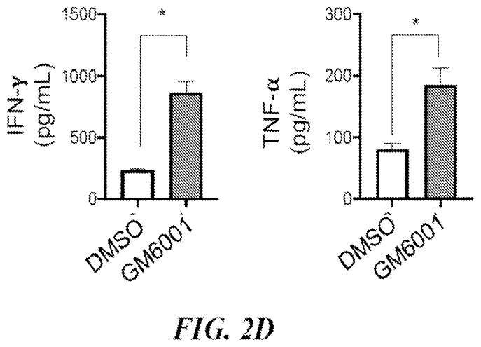

In this example, mesothelin CAR constructs were generated and their efficacy against mesothelin expressing cells was tested. Accordingly, this example demonstrates that treatment of Nomo-1 cells with GM6001 resulted in an increased level of cell surface mesothelin (FIG. 2A) with a corresponding reduction in soluble mesothelin in the cultured supernatant (FIG. 2B), suggesting that GM6001 treatment may stabilize and prevent the release of mesothelin on the cell surface. GM6001 treatment during co-culture of Nomo-1 and CAR T cells enhanced the cytolytic activity and cytokine production of CAR T cells (FIGS. 2C-2D). GM6001 treatment did not significantly impact cell viability of Nomo-1 cells in the absence of CAR T cells (data not shown).

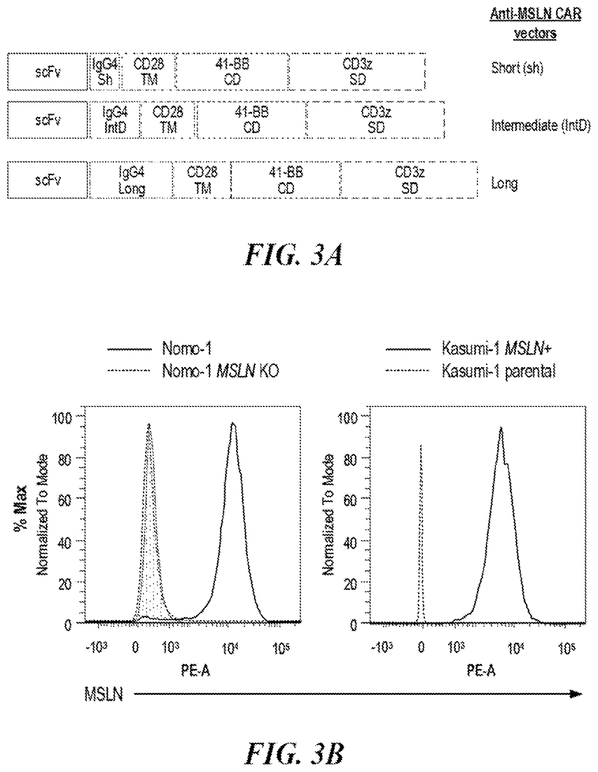

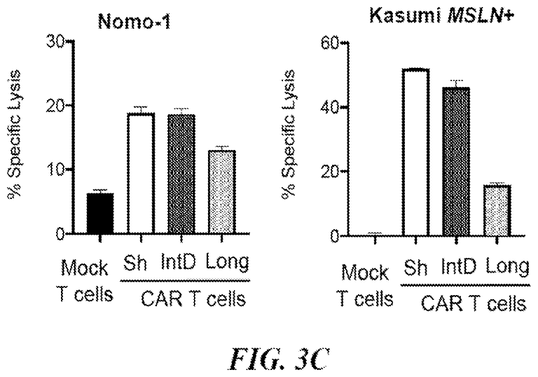

MSLN CAR constructs. MSLN-directed CARs were generated by inserting the single-chain variable fragment (scFv) derived from SS1 into the CAR vectors composed of IgG4 hinge, CD28 transmembrane, 41-BB co-stimulatory, and CD3zeta stimulatory domains (FIG. 3A).

AML cell lines and GM6001 treatment. Nomo-1 and Kasumi-1 were obtained from ATCC and maintained in RPMI with FBS (10% for Nomo-1, 20% for Kasumi-1) and L-glutamine (2 mM). MSLN is expressed on the cell surface of Nomo-1 cells but not Kasumi-1 cells (FIG. 3B). The Kasumi-1 MSLN+ cell line was engineered by transducing Kasumi-1 cells with a lentivirus encoding MSLN driven by the ELF1a promoter. Nomo-1 cells were treated with GM6001 (50 uM) or DMSO control for 48 hr prior to evaluation of surface mesothelin by flow cytometry, soluble mesothelin in the culture supernatant by ELISA and cytotoxicity with MSLN CAR T cells.

AML xenografts. Nomo-1 and Kasumi MSLN+ cells were transplanted into NSG mice at 10⁶ per mouse. MSLN-directed CART cells or mock transduced T cells were infused 1 week following Nomo-1 injection and 2 weeks following Kasumi-1 MSLN+ injection. Leukemic burden was measured by bioluminescence IVIS imaging weekly until mice developed symptoms (hunchback, persistent weight loss, fatigue, or hind-limb paralysis) or until an experimental endpoint of 16 weeks post leukemia injection.

From primary patient samples, MSLN expression was verified by RT-PCR and confirmed mesothelin surface protein expression on leukemic blasts by flow-cytometry as well as detected soluble mesothelin in the plasma by ELISA. The V_{H} and VL sequences from Amatuximab were used to create the scFv domain of the standard CAR (41-BB and CD3Zeta). For in vivo CAR T study, Nomo-1 cells, which express endogenous level of MSLN, and Kasumi-1 cells engineered to express MSLN with a lentivirus construct (Kasumi-1 MSLN+) were transplanted into NSG mice. Mock transduced MSLN-directed CAR T cells were infused 1 week (Nomo-1) and 2 weeks (Kasumi-1 MSLN+) following leukemic cell injection. Leukemic burden was measured by bioluminescence IVIS imaging weekly. For in vitro study, Nomo-1 cells were treated with GM6001 (50 uM), a metalloprotease inhibitor, or DMSO control for 48 hr prior to evaluation of surface mesothelin by flow cytometry and soluble mesothelin in the culture supernatant by ELISA.

Without intending to be bound by any particular theory, it is thought that the distance between scFv domain and the T cell surface affects CAR function. As such, the spacer region of the CAR was optimized, and it was determined that the MSLN CAR with the short hinge domain conferred superior cytotoxicity over the CARs with intermediate and long spacer (FIG. 3C).

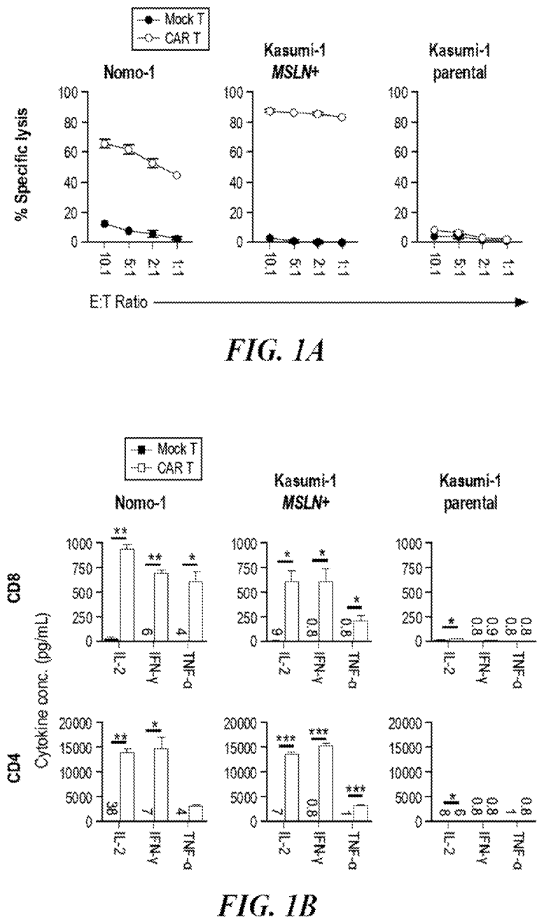

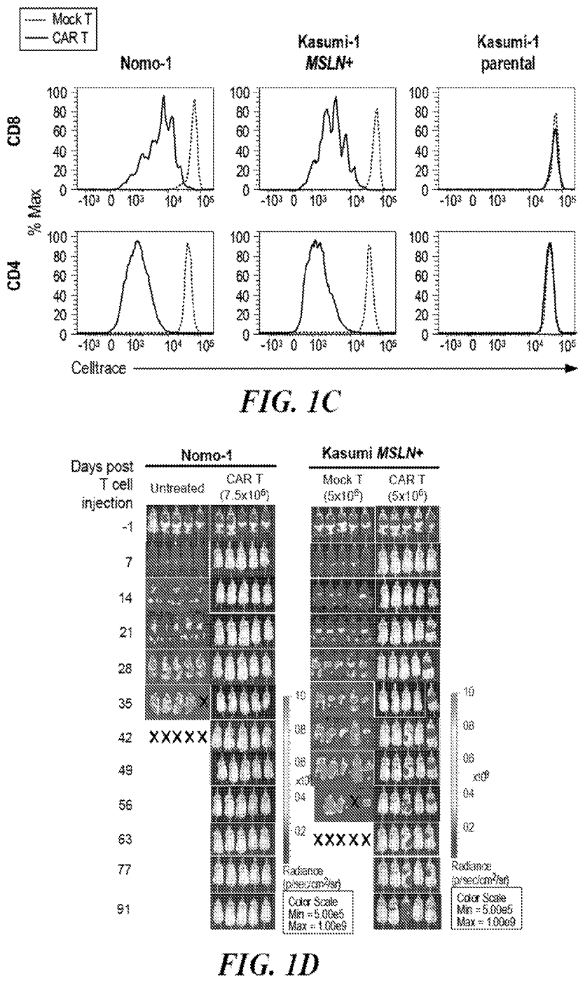

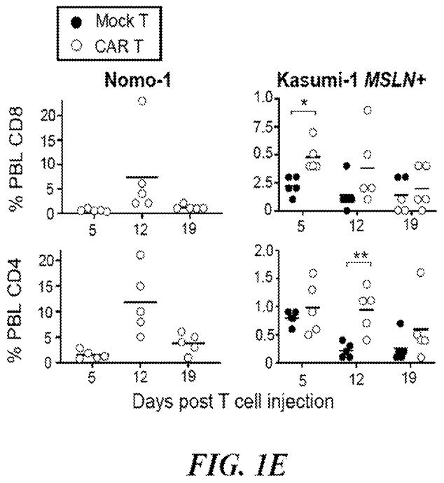

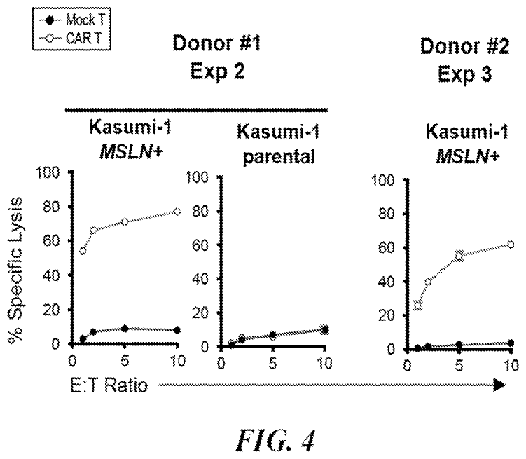

Using the short MSLN CAR construct, the antigen-specific reactivity of MSLN CAR T cells against AML cell lines, including Nomo-1 cells which naturally express MSLN, Kasumi-1 cells engineered to express MSLN (Kasumi-1 MSLN+), and Kasumi-1 parental cells which were used as a negative control were evaluated. MSLN CAR T cells demonstrated potent cytolytic activity against Nomo-1 and Kasumi-1 MSLN+ cells while mock transduced T cells had background activity (FIG. 1A, FIG. 4). The anti-leukemia killing capacity was antigen specific as the viability of Kasumi-1 parental cells was unaffected following coincubation with MSLN CAR or mock transduced T cells (FIG. 1A).

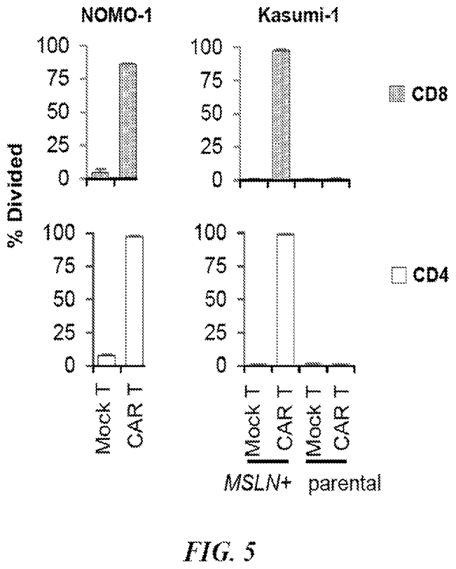

To further evaluate the antigen-specific reactivity of MSLN CAR T cells, cytokine production and cell proliferation of MSLN CAR T cells following coculture with target cells were measured. Both CD4 and CD8 MSLN CAR T cells produced higher levels of interferon-gamma (INF-g), tumor-necrosis factor-alpha (TNF-a), and interleukin-2 (IL-2) compared to mock transduced T cells in the presence of Nomo-1 and Kasumi-1 MSLN+ cells (FIG. 1B). However, when co-incubated with Kasumi-1 parental cells, MSLN CAR T cells produced background levels of INF-g, TNF-a, and IL-2 similar to mock transduced T cells (FIG. 1B). The specificity of MSLN CAR T cells was confirmed by CFSE cell proliferation assay. Both CD8 and CD4 MSLN CART cells rapidly expanded when co-incubated with Nomo-1 and Kasumi-1 MSLN+ cells but lacked proliferative potential in the presence of Kasumi-1 parental cells (FIG. 1C, FIG. 5). These results indicate highly specific reactivity of MSLN CAR T cells against MSLN-positive AML cells.

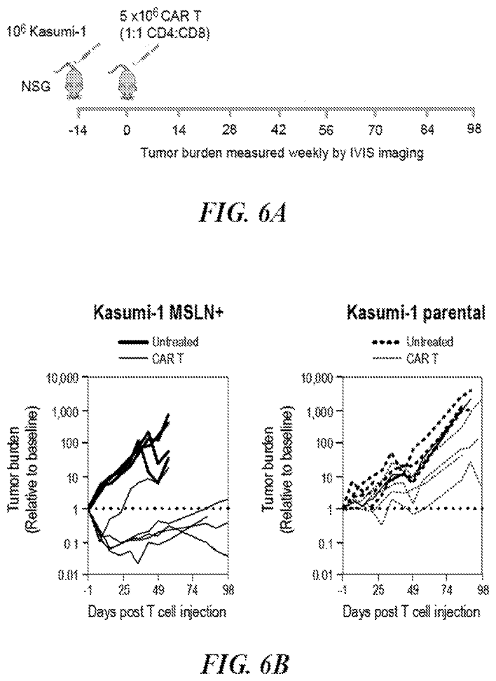

The ability of MSLN CAR T cells to eradicate AML was determined in vivo. Luciferase-expressing Nomo-1 and Kasumi-1 MSLN+ cells were injected into NSG mice and infused with MSLN CAR T cells once leukemic engraftment was established. MSLN CAR T cells induced leukemia clearance within a week of CAR T cell infusion in both Nomo-1 and Kasumi-1 MSLN+ xenograft models (FIG. 1D). The leukemia clearance was maintained in the Nomo-1 mice for the entire duration of the study (120 days) with no signs of leukemia at necropsy, whereas the untreated mice had a median survival of 42 days (FIG. 1D, left). Along the same line, a majority of Kasumi-1 MSLN+ mice given CAR T treatment had no detectable leukemia by bioluminescent imaging through day 35 post T cell injection and survived until the end of the study (compared to the median survival of 56 days in mice receiving mock transduced T cells, FIG. 1D, right). The in vivo activity of MSLN CAR T cells was antigen specific as the CAR T cells did not limit the progression of Kasumi-1 parental cells in NSG mice (FIGS. 6A-6B). Without intending to be bound by any particular theory, it is thought that the robust anti-leukemic activity of MSLN CAR T cells observed in Nomo-1 and Kasumi-1 MSLN+ xenografts may be attributed to the rapid expansion of CAR-positive CD4 and CD8 in vivo as quantified by flow cytometric analysis of mouse peripheral blood (FIG. 1E).

In vivo cytotoxicity of CAR T cells against Nomo-1 and Kasumi-1 MSLN+ AML models demonstrated potent, target-dependent tumor killing. After 1 and 2 weeks post CAR T infusion, leukemic cells were eradicated in both Nomo-1 (p<0.0005, week 2) and Kasumi-1 MSLN+ xenografts (p<0.005 at week 2, FIGS. 1A-16). Mesothelin undergoes shedding at the cell membrane as a result of ADAM17-mediated cleavage. Blocking ADAM17 activity with GM6001 in Nomo-1 cells led to increased cell surface mesothelin (FIG. 1C) with a corresponding reduction in the shed form (FIG. 1D), suggesting that GM6001 treatment stabilizes mesothelin on the cell surface. Furthermore, GM6001 treatment during co-culture of Nomo-1 and CAR T cells enhanced cytolytic activity of CAR T cells (FIG. 1E). GM6001 treatment did not significantly impact cell viability of Nomo-1 cells in the absence of CAR T cells (data not shown).

The example demonstrates that mesothelin is a viable therapeutic target and may be a potential diagnostic biomarker in AML. MSLN CAR T cells were highly effective in eliminating MSLN-positive AML cells in vitro and in vivo. Without intending to be bound by any particular theory, it is thought that shedding contributes to the loss of cell surface mesothelin antigen and provides a source of soluble mesothelin that may interfere with antibody-based therapies, including CAR T cells. In some embodiments, modulating MSLN shedding by inhibiting ADAM17-mediated cleavage resulted in stabilized mesothelin and improved CAR T cell functionality. MSLN CAR T cells may be tested in clinical trials for AML. Without intending to be bound by any particular theory, it is thought that inhibiting MSLN shedding may improve CAR T efficacy.

### CONCLUSION

The above detailed description of embodiments of the technology is not intended to be exhaustive or to limit the technology to the precise forms disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known components and functions have not been shown or described in detail to avoid unnecessarily obscuring the description of the embodiments of the technology. Where the context permits, singular or plural terms may also include the plural or singular term, respectively. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

### REFERENCES

1. Awuah, P., Bera, T. K., Folivi, M., Chertov, O. & Pastan, I. Reduced Shedding of Surface Mesothelin Improves Efficacy of Mesothelin-Targeting Recombinant Immunotoxins. Mol Cancer Ther 15, 1648-1655, doi:10.1158/1535-7163.MCT-15-0863 (2016).
2. Pastan, I. & Zhang, Y. Modulating mesothelin shedding to improve therapy. Oncotarget 3, 114-115, doi:10.18632/oncotarget.445 (2012).
3. Pak, Y., Zhang, Y., Pastan, I. & Lee, B. Antigen shedding may improve efficiencies for delivery of antibody-based anticancer agents in solid tumors. Cancer Res 72, 3143-3152, doi:10.1158/0008-5472.CAN-11-3925 (2012).
4. Pak, Y., Pastan, I., Kreitman, R. J. & Lee, B. Effect of antigen shedding on targeted delivery of immunotoxins in solid tumors from a mathematical model. PLoS One 9, e110716, doi:10.1371/journal.pone.0110716 (2014).
5. Zhang, Y., Chertov, O., Zhang, J., Hassan, R. & Pastan, I. Cytotoxic activity of immunotoxin SS1P is modulated by TACE-dependent mesothelin shedding. Cancer Res 71, 5915-5922, doi:10.1158/0008-5472.CAN-11-0466 (2011).
6. Adusumilli, P. S. et al. Regional delivery of mesothelin-targeted CAR T cell therapy generates potent and long-lasting CD4-dependent tumor immunity. Sci Transl M ed 6, 261 ra151, doi:10.1126/scitranslmed 0.3010162 (2014).
7. Garcia-Guerrero, E., Sierro-Martinez, B. & Perez-Simon, J. A.

Overcoming Chimeric Antigen Receptor (CAR) Modified T-Cell Therapy Limitations in Multiple Myeloma. Front Immunol 11, 1128, doi: 10.3389/fimmu.2020.01128 (2020).
8. Cartellieri, M. et al. Chimeric antigen receptor-engineered T cells for immunotherapy of cancer. J Biomed Biotechnol 2010, 956304, doi:10.1155/2010/956304 (2010).
9. Grigoriadis, G. & Whitehead, S. CD138 shedding in plasma cell myeloma. Br J Haematol 150, 249, doi:10.1111/j.1365-2141.2010.08203.x (2010).
10. Lichtenthaler, S. F., Lemberg, M. K. & Fluhrer, R. Proteolytic ectodomain shedding of membrane proteins in mammals-hardware, concepts, and recent developments. EMBO J 37, doi:10.15252/embj.201899456 (2018).
11. Van Schaeybroeck, S. et al. Oncogenic Kras promotes chemotherapy-induced growth factor shedding via ADAM17. Cancer Res 71, 1071-1080, doi: 10.1158/0008-5472.CAN-10-0714 (2011).
12. Kyula, J. N. et al. Chemotherapy-induced activation of ADAM-17: a novel mechanism of drug resistance in colorectal cancer. Clin Cancer Res 16, 3378-3389, doi: 10.1158/1078-0432.CCR-10-0014 (2010).

We claim:

1. A chimeric antigen receptor (CAR) that binds to at least one epitope of mesothelin.

2. The CAR of claim 1, wherein the CAR comprises a signal peptide, a binding domain specific to mesothelin, a hinge domain, a transmembrane domain, a costimulatory domain, and/or an effector domain.

3. The CAR of claim 2, wherein the signal peptide comprises a GM-CSFR signal peptide.

4. The CAR of claim 2, wherein the binding domain comprises an scFv.

5. The CAR of claim 4, wherein the scFv comprises a light chain variable region (V_{L}) having an amino acid sequence that is at least 75%, 80%, 85%, 90%, 91%, 92%, 93%, 94%, 95%, 96%, 97%, 98%, 99%, or 100% identical to SEQ ID NO: 1.

6. The CAR of claim 4, wherein the scFv comprises a heavy chain variable region (V_{H}) having an amino acid sequence that is at least 75%, 80%, 85%, 90%, 91%, 92%, 93%, 94%, 95%, 96%, 97%, 98%, 99%, or 100% identical to SEQ ID NO: 3.

7. The CAR of any one of claims 4-6, wherein the scFv comprises a (G₄S)₄ linker connecting a V_{L} and a V_{H}.

8. The CAR of any one of claims 4-7, wherein the scFv comprises an amino acid sequence that is at least 75%, 80%, 85%, 90%, 91%, 92%, 93%, 94%, 95%, 96%, 97%, 98%, 99%, or 100% identical to any one of SEQ ID NOs: 1-3.

9. The CAR of claim 2, wherein the hinge domain comprises an IgG4 hinge domain.

10. The CAR of claim 2, wherein the transmembrane domain comprises a CD28 transmembrane domain.

11. The CAR of claim 2, wherein the costimulatory domain comprises a 4-1BB costimulatory domain.

12. The CAR of claim 2, wherein the effector domain comprises a CD3 effector domain.

13. The CAR of claim 2, wherein the CAR further comprises a spacer between the hinge domain and the transmembrane domain.

14. The CAR of claim 13, wherein the spacer comprises an amino acid sequence that is at least 75%, 80%, 85%, 90%, 91%, 92%, 93%, 94%, 95%, 96%, 97%, 98%, 99%, or 100% identical to SEQ ID NO: 7 or SEQ ID NO: 8.

15. The CAR of claim 2, wherein the CAR further comprises a polypeptide marker.

16. The CAR of claim 2, wherein the polypeptide marker comprises a truncated form of CD19 (CD19t) comprising an amino acid sequence set forth in SEQ ID NO: 12.

17. The CAR of claim 16, wherein the CD19t is separated from the CAR by a T2A sequence comprising an amino acid sequence set forth in SEQ ID NO: 11.

18. The CAR of claim 1, wherein the CAR comprises an amino acid sequence that is at least 75%, 80%, 85%, 90%, 91%, 92%, 93%, 94%, 95%, 96%, 97%, 98%, 99%, or 100% identical to any one of SEQ ID NOs: 13-15.

19. An isolated polynucleotide encoding the CAR of any one of claims 1-18.

20. The polynucleotide of claim 19, wherein the polynucleotide is in a vector.

21. A T cell, a natural killer (NK) cell, or an NKT cell expressing the CAR of any one of claims 1-18 or comprising the polynucleotide of claim 19 or 20.

22. A method of treating and/or preventing a cancer associated with mesothelin expression in a subject in need thereof, comprising administering to the subject a therapeutically effective amount of the CAR of any one of claims 1-18, the polynucleotide of claim 19 or 20, or the T cell, NK cell, or NKT cell of claim 21.

23. The method of claim 22, wherein the cancer is acute myeloid leukemia (AML).


SEQUENCE LISTING (15 sequences)

SEQ ID NO: 1 - protein, 106 aa, Artificial Sequence
  SS1 VL
DIELTQSPAIMSASPGEKVTMTCSASSSVSYMHWYQQKSGTSPKRWIYDTSKLASGVPGRFSGSGSGNSYSLTISSVEAEDDATYYCQQWSGYPLTFGAGTKLEIK

SEQ ID NO: 2 - protein, 20 aa, Artificial Sequence
  (G4S)4
GGGGSGGGGSGGGGSGGGGS

SEQ ID NO: 3 - protein, 119 aa, Artificial Sequence
  SS1 VH
QVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSS

SEQ ID NO: 4 - protein, 22 aa, Artificial Sequence
  GM-CSFR signal peptide
MLLLVTSLLLCELPHPAFLLIP

SEQ ID NO: 5 - protein, 112 aa, Artificial Sequence
  CD3zeta effector domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 6 - protein, 6 aa, Artificial Sequence
  IgG4 hinge domain
ESKYGP

SEQ ID NO: 7 - protein, 113 aa, Artificial Sequence
  Intermediate spacer
PCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 8 - protein, 222 aa, Artificial Sequence
  Long spacer
PCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 9 - protein, 27 aa, Artificial Sequence
  CD28 transmembrane domain
MFWVLVVVGGVLACYSLLVTVAFIIFW

SEQ ID NO: 10 - protein, 42 aa, Artificial Sequence
  4-1BB costimulatory domain
KRGRKKLLYIFKQPFMRPVQTTQEEDGCSCRFPEEEEGGCEL

SEQ ID NO: 11 - protein, 24 aa, Artificial Sequence
  T2A
LEGGGEGRGSLLTCGDVEENPGPR

SEQ ID NO: 12 - protein, 323 aa, Artificial Sequence
  Truncated CD19 (CD19t)
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCVPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKR

SEQ ID NO: 13 - protein, 801 aa, Artificial Sequence
  Short mesothelin CAR
MLLLVTSLLLCELPHPAFLLIPDIELTQSPAIMSASPGEKVTMTCSASSSVSYMHWYQQKSGTSPKRWIYDTSKLASGVPGRFSGSGSGNSYSLTISSVEAEDDATYYCQQWSGYPLTFGAGTKLEIKGGGGSGGGGSGGGGSGGGGSQVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSSESKYGPMFWVLVVVGGVLACYSLLVTVAFIIFWKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCVPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKR

SEQ ID NO: 14 - protein, 914 aa, Artificial Sequence
  Intermediate mesothelin CAR
MLLLVTSLLLCELPHPAFLLIPDIELTQSPAIMSASPGEKVTMTCSASSSVSYMHWYQQKSGTSPKRWIYDTSKLASGVPGRFSGSGSGNSYSLTISSVEAEDDATYYCQQWSGYPLTFGAGTKLEIKGGGGSGGGGSGGGGSGGGGSQVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSSESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCVPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKR

SEQ ID NO: 15 - protein, 1023 aa, Artificial Sequence
  Long mesothelin CAR
MLLLVTSLLLCELPHPAFLLIPDIELTQSPAIMSASPGEKVTMTCSASSSVSYMHWYQQKSGTSPKRWIYDTSKLASGVPGRFSGSGSGNSYSLTISSVEAEDDATYYCQQWSGYPLTFGAGTKLEIKGGGGSGGGGSGGGGSGGGGSQVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCVPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKR